(19) United States
(12) Patent Application Publication
(10) Pub. No.: US 2009/0253160 A1
(43) Pub. Date: Oct. 8, 2009

(54) NOVEL METHOD OF PREDICTING PIG LITTER SIZE BY EVALUATING SEMEN

(75) Inventors: Myung-Geol Pang, Seoul (KR); Shin-Ae Oh, Anseong-si (KR)

Correspondence Address: GREENLEE WINNER AND SULLIVAN P C, 4875 PEARL EAST CIRCLE, SUITE 200, BOULDER, CO, 80301, US

(73) Assignee: Chung-Ang University Industry-Academy Coorperation Foundation, Seoul (KR)

(21) Appl. No.: 12/234,419

(22) Filed: Sep. 19, 2008

(30) Foreign Application Priority Data
Apr. 4, 2008 (KR) 10-2008-31755

(51) Int. Cl. C12Q 1/02

(52) U.S. Cl. 435/29

(57) ABSTRACT

Provided is a method of predicting pig litter size by evaluating semen, and more particularly, a method of predicting pig litter size using an in-vitro sperm penetration assay (SPA).

This is a novel method of predicting in vivo fertilization using a sperm penetration assay in vitro after optimizing each step of the procedure. Thus, it can more accurately predict excellent individuals for producing smaller or larger litters.

### CROSS-REFERENCE TO RELATED APPLICATION

This application claims the benefit of Korean Patent Application No. 2008-0031755 filed 04, 04, 2008, the disclosure of which is hereby incorporated herein by reference in its entirety.

### BACKGROUND OF THE INVENTION

1. Field of the Invention

The present invention relates to a novel method of predicting pig litter size by evaluating semen, and more particularly, to a method of predicting pig litter size by a sperm penetration assay.

2. Description of the Related Art

Porcine artificial insemination is a means to stimulate improvement in pig breeding, increase fertility, decrease breeding costs and manpower, and prevent spread of diseases. Thus, the rate of its application has increased about three times over the last 15 years.

However, artificial insemination lacks productivity compared to its high application rate, and the results of its application to breeding widely vary. That is, while the rate of its application has increased, artificial insemination has not been used effectively to improve pig breeding.

Due to poor quality of semen commercially provided by the artificial insemination center, which focuses just on quantitative improvement of artificial insemination, pig breeders are increasingly turning back to natural insemination. The isolation and provision of high-quality semen is crucial to efficient application of artificial insemination.

However, the quality of sperm is evaluated in a very subjective way, and a test for evaluating sperm quality conducted by the artificial insemination center is just a sperm motility test. The sperm motility test alone does not enable accurate prediction of potency, and thus cannot be used as an indicator of sperm quality. The artificial insemination center supplies domestic and imported boars to domestic pig breeders. However, the demand for domestic boars exceeds their supply, and thus swine capable of producing larger litters are needed. However, if pig semen in imported from overseas in order to obtain more productive swine, since there is no objective index for evaluating the quality of pig semen, low quality semen may be reluctantly purchased.

Based on a million domestic sows, each sow farrows about 2.5 times per year. It is estimated that two more piglets can be produced per minute by a conventional technique, which results in a total profit of 25 billion won per year (2 piglets×2.5 times×50,000 won×1 million pigs). It is also estimated that when the artificial insemination center adopts this technique, an additional profit of 2 or more billion won can be made by discriminating between highly potent and less potent semen. Moreover, reliable prediction of litter size would be conducive to stable pig production.

Meanwhile, there are conventional assays for evaluating boar sperm fertility including an in-vitro fertilization assay using zona-intact oocytes (Martinez et al., 1993; Matas et al., 1996; and Gadea et al., 1998) or in-vitro matured oocytes (Xu et al., 1996) in a germinal vesicle stage, a hemizona binding assay (Fazeli et al., 1995), and sperm binding to zona-intact porcine oocytes or stored porcine oocytes (Lynham and Harrison, 1998). An in-vitro insemination system is one effective method of predicting pig sperm fertility. Xu et al. ascertained an effective relationship between the number of sperm cells binding to an ovum and litter size. Further, a homologous in vitro penetration (hIVP) assay using zona-intact porcine oocytes is one of the effective methods of evaluating boar sperm fertility. The zona-intact porcine oocytes exhibit similar penetration to oocytes that have been matured in vitro or ovulated (Gadea and Matas, 2000). Gadea and Matas (2000) determined a cut-off value of a normal range as 75% to predict in-vivo insemination by hIVP. The accuracy of this method is 74.17%. Although it was reported by Berger et al. (1996) that there is no relationship between historic fertilization and sperm-zona binding ability, Gadea and Mates proved the relationship between sperm penetration to cytoplasm of zona-free hamster oocytes and historic pregnancy. Therefore, the values presented by Gadea and Mates are considered to be very closely predictive values.

The prediction of sperm fertility is very important in breeding animals when artificial insemination is applied (Gadea, 2005). Conventional semen analyses generally provide quantitative information on the semen itself and involve an evaluation of the percentage of motile sperm cells, the percentage of sperm cells with normal morphology, and the concentration in a unit dose. While these assays provide valuable quantitative data, they yield no information concerning the functional competence of the individual sperm cells (Chang et al., 1990; Johnson et al., 1990; and Petrunkina et al., 2007).

To compensate for the shortcomings of conventional semen analyses, some tests that evaluate sperm function and fertility are economically important and have been developed for animal species and humans. However, they have been developed to detect only one of a few of the events involved in the complex fertilization process, for example, the acquirement of hyperactivated movement, an intact acrosome, the ability to fertilize, normal DNA status, the ability to bind to the zonal pellucida, and fusion with the oocyte. These evaluations do not perfectly address sperm function and their clinical value in predicting fertility (Lewis, 2007). Therefore, for most of these tests, relatively little correlation has been found with sperm function and fertility (Gadea and Matas, 2000, Johnson et al., 2000; Rodriquez-Martinez, 2003). There is an urgent need of new sperm function parameters that correlate better with in vivo fertility (Petrunkina et al., 2007).

One obvious method of assessing sperm fertility would be to ascertain whether sperm can actually fertilize homologous eggs in vitro (Xu et al., 1996). However, contradictory results have been observed in cattle with respect to the relationship between in vitro penetration rates and in vivo sperm fertility (Kruip et al., 1992). These contradictory results indicate that homologous IVF methods need to be simplified and standardized before they can be used to evaluate the penetrating ability of mammalian sperm cells (Roca et al., 1998). It has been reported that zona-intact immature oocytes show a similar degree of penetrability to that seen in oocytes that have been matured or ovulated in vitro (Matas et al., 1996). The use of immature oocytes in a hIVP assay of boar sperm fertility would facilitate the collection of female gametes, thus reducing the time required for in vitro maturation. The hIVP has previously been used as a means of testing the penetration ability of boar semen (Martinez et al., 1996). The results from the hIVP assay have been correlated with in vivo fertility of liquid boar semen (Gadea et al., 1998; Martinez et al., 1998). However, they are of limited value in predicting the fertilizing ability of sperm.

Many investigators have reported modifications of the sperm penetration assay (SPA) protocol using zona-free hamster ova to evaluate sperm fertility. A relatively common assay used for fertility determination in many laboratories is a xenogeneric sperm penetration test using human (Chang et al., 1990; Sofikitis et al., 2000; and Wiland et al, 2000), bovine (Eaglesome and Garcia, 1990), or rabbit (Rajeev and Reddy, 2004) sperm cells and hamster oocytes. While the SPA has received widespread attention as a test of sperm fertility, the wide range of penetration levels that constitute normal fertility, interassay variability and lack of quality control are inherent problems of this bioassay system.

Capacitation and the subsequent acrosome reaction are essential for the in vitro penetration assay. Therefore, to improve the accuracy and reliability of in vitro sperm fertility tests, it is necessary to optimize each procedure and to include adequate quality control. Accordingly, the present inventors developed and optimized a novel method of predicting a fertility rate in boars using the in vitro penetration assay. To increase sensitivity, each step in the procedure was optimized, and to increase accuracy in predicting pig litter size, SPA using quality control was applied.

This study was supported by “Biogreen 21 Program” of the Ministry for Food, Agriculture, Forestry and Fisheries, Korea.

### SUMMARY OF THE INVENTION

An object of the invention is to provide a method of predicting pig litter size by evaluating semen. More particularly, an object of the invention is to provide an optimized method of predicting a sperm fertility rate using an in-vitro penetration assay, each step of the method being optimized and performed under quality control.

In one aspect, the present invention provides a method of predicting boar sperm fertility, including incubating sperm cells from semen extracted from the boar in a heparin-treated medium, co-incubating the sperm cells with zona-free hamster oocytes in a heparin-treated medium, and counting the numbers of penetrating sperm cells and pronuclei per ovum to analyze in-vitro sperm penetration.

The prediction of sperm fertility may be performed by prediction of litter size.

The litter size may be predicted as 8 or fewer piglets when SFI given by Formula I is equal to or smaller than 1.2, and 10 or more piglets when SFI given by Formula I is more than 2.5.

SFI={the mean number of penetrated sperm per ovum(the number of enlarged sperm heads+the number of decondensed sperm heads)+(the number of pronuclei per ovum×2)}/the number of ova <Formula 1>

The present method may further include simultaneously treating frozen bovine semen by the same procedure as described above to be used as an internal control for the prediction of boar sperm fertility, and to detect errors in the test.

The heparin-treated medium may be formed by treating a medium supplemented with inactivated fetal calf serum, sodium pyruvate, D-glucose, calcium lactate, penicillin G and streptomycin sulfate with heparin, and the sperm cells may be incubated at 39° C. for 30 minutes.

The sperm cells may be diluted to a concentration of 2×10⁶ cells/ml in a heparin medium for co-incubation. Preferably, the sperm cells may be co-incubated at 39° C. for 2 to 4 hours in a 5% CO₂ atmosphere.

For the present invention, sows inseminated twice per estrus by very well-trained technicians are used to exclude all possible variables. To increase predictive accuracy, all possible variables related to the technician, sow parity, and number of inseminations during estrus, are excluded.

Then, conditions for maximum sperm penetration (wide penetration range) are established to increase discrimination between boars showing good and bad sperm fertility by optimizing SPA and testing an increase in sensitivity in each step of the SPA.

That is, the maximum penetration of heparin-treated sperm cells is yielded in zona-free hamster oocytes and immature porcine oocytes. The zona-free hamster oocytes show a significantly higher penetration rate than the immature porcine oocytes, and the comparison of SCl and SFI demonstrates that SFI shows a wider range of the spectrum.

Therefore, the present invention provides a method of predicting porcine sperm fertility, which includes incubating sperm cells obtained from semen extracted from a boar in a heparin-containing medium, co-incubating the cultured sperm cells with zona— free hamster oocytes in the heparin-containing medium, and counting the numbers of sperm cells penetrated into an ovum and pronuclei to analyze in-vitro sperm penetration.

The method may further include excluding or identifying errors during the in-vitro sperm penetration assay applied to frozen bovine semen samples, which may be used as controls for predicting porcine sperm fertility.

In one embodiment of the present invention, to exclude inherent problems of SPA, such as a lack of quality control and errors in analyses, two frozen bovine semen samples having medium fertility were used. No significant statistical difference was detected when quality control was performed eight times. This result showed that the SPA used herein produced uniform results, and the frozen bovine sperm samples were effective internal controls for the SPA.

The prediction of fertility may be to predict pig litter size.

The litter size may be predicted as 8 or fewer piglets when SFI given by Formula 1 is equal to or smaller than 1.2, and 10 or more piglets when SFI given by Formula 1 is more than 2.5.

SFI={the mean number of penetrated sperm per ovum(the number of enlarged sperm heads+the number of decondensed sperm heads)+(the number of pronuclei per ovum×2)}/the number of ova <Formula 1>

In another embodiment of the present invention, a significant relationship was found between SFI and historic mean litter size, but interestingly, no significant relationship was found between SFI and farrowing rate.

Further, previous results obtained by other analyses reveal a relationship between litter size and boar zona binding ability, not farrowing rate (Braundmeir et al., 2004). Xu et al. (1998) reported that litter size may correspond more closely to fertility than farrowing rate, and Braundmeier (2004) disclosed a possible explanation of this phenomenon. However, the estimation of farrowing rate has just two possible results: pregnancy and non-pregnancy.

On the other hand, the prediction of fertility may provide more accurate values for boar fertility by predicting litter size.

In addition, the present invention determines a normal range for optimized SPA. Thus, a lower limit of SFI is determined as 1.2 for smaller litter sizes (8 or fewer piglets), and a lower limit of SFI is determined as 2.5 for larger litter sizes (10 or more piglets). It is confirmed that overall accuracies of the analyses are 92% for the smaller litter size and 96% for the larger litter size, which are significantly higher than conventional predictive accuracy (75%).

### BRIEF DESCRIPTION OF THE DRAWINGS

The above and other objects, features and advantages of the present invention will become more apparent by describing certain exemplary embodiments in detail with reference to the attached drawings.

FIG. 1 shows an effect of variables on the results of SPA. Different capacitation methods and ovum sources are compared, and data are expressed as mean (±SE) SCI/SFI. 80 to 90 immature porcine oocytes were used per treatment group, and 30 to 33 zona-free hamster oocytes were used per treatment group in three individual experiments (TCM 199=control, TYB-4=treatment of TYM at 4° C., 24 hr, TYB-24=treatment of TYB at 24° C., 30 min, Heparin=treatment of heparin in culture medium).

In FIG. 1, a, b, c SCI values for TCM 199, TYB-4, TYB-24 and heparin in immature porcine oocytes have significant differences, and in zona-free hamster oocytes have significant differences by ANOVA (p<0.05).

Moreover, in FIG. 1, e and f SFI values for TCM 199, TYB-4, TYB-24 and heparin in immature porcine oocytes have significant differences, and in zona-free hamster oocytes have significant differences by ANOVA (p<0.05).

*SCI and SFI groups are significantly different from each other (p<0.05).

FIG. 2 is a quality control plot for a sperm penetration assay. Frozen bovine semen for medium penetration was used to monitor interassay variables (EN: enlarged sperm, DC: decondensed sperm, PN: formed pronucleus, SFI: an index for sperm fertility).

FIG. 3a shows a relationship between SPA and historic litter size. A significant correlation (r=0.726, p<0.05) was detected between SFI and litter size for 24 boars. FIG. 3b shows a relationship between SPA and historic farrowing rate. No significant correlation (r=0.140) was detected between SFI and farrowing rate. That is, SFI shows a significant relationship to historic mean litter size (r=0.726, p<0.05), but no significant relationship to historic farrowing rate.

FIG. 4 shows the determination of a lower limit of normal litter size for optimized SPA. The lower limit in a spectrum of the normal litter size is defined as 8 or more.

FIG. 5 shows the determination of a lower limit of smaller litter size for optimized SPA. A lower limit of larger litter size is determined as 10 or more.

### DETAILED DESCRIPTION OF THE INVENTION

The present invention will now be described more fully hereinafter with reference to exemplary embodiments and experimental examples shown in the attached drawings. This invention may, however, be embodied in different forms and should not be construed as limited to the embodiments and examples set forth herein.

### Exemplary Embodiment 1

### Materials and Methods

1-1: Preparation of Medium and Buffer

(1) Medium

A medium 199 with Earle's salts was supplemented with 10% heat-inactivated fetal calf serum (v/v), 0.91 mM sodium pyruvate, 3.05 mM D-glucose, 2.92 mM calcium lactate, 50 IU/l penicillin G. and 30 ug/ml streptomycin sulfate. Heparin (10 ug/ml) was added to the basic medium for heparin treatment.

(2) Tris Yolk Buffer (TYB)

211 mM TES (N-tris methyl-2-aminethane sulfonic acid), 96 mM Tris (hydroxymethylaminomethane), 11 mM dextrose and 20% fresh egg yolk (v/v) was added to distilled water, and the resulting solution was adjusted to final pH 7.4 and an osmolarity of 300mOsmo/kg.

1-2: Preparation of Sperm Cells

(1) Preparation of Semen Sample for Optimization

To calculate the accuracy of the optimized hIVP and SPA and to determine the normal range, commercial semen samples were used. Whole semen samples from Duroc boars were collected at weekly intervals by a gloved hand technique and filtered through cotton gauze into a pre-warmed flask at 25° C. Mobility of ejaculates was more than 80%. Boar semen samples were diluted with Beltsville thawing solution (BTS) and kept at 17° C.

(2) Semen Samples for Sperm Penetration Assay

To evaluate fertility, sows having more than five litters from respective boars were selected (on average, 22.46 inseminations and 8.96 litters were evaluated per boar).

Fertility data shown in Table 1 were obtained from Darby Genetics, Inc. (Korea). The farrowing rate and litter size were used as in-vivo fertility parameters. The average farrowing rates were calculated for each boar as the percentage of sows that successfully farrowed. The average litter size for each boar was calculated as the total number of piglets born (alive and dead) from each farrowing, averaged for all farrowings (Braundmeier et al., 2004).

TABLE 1
In vivo fertility data from each of 24 boars

| | Number of sows | | | | |
|---|---|---|---|---|---|
| | (sow-female pig | Farrowing | | | |
| No. | experienced in farrowing) | rate(%) | | Litter size | |
| A | 27 | 100.00 | ±.00 | 5.50 | ±.39 |
| B | 6 | 100.00 | ±.00 | 6.38 | ±.24 |
| C | 26 | 100.00 | ±.00 | 6.80 | ±.60 |
| D | 42 | 100.00 | ±.00 | 7.50 | ±.94 |
| E | 12 | 75.44 | ±.61 | 7.69 | ±.69 |
| F | 8 | 100.00 | ±.00 | 7.70 | ±.12 |
| G | 74 | 33.78 | ±.03 | 7.81 | ±.64 |
| H | 153 | 58.17 | ±.66 | 8.19 | ±.42 |
| I | 82 | 37.80 | ±.98 | 8.58 | ±.58 |
| J | 98 | 40.82 | ±.14 | 8.75 | ±.61 |
| K | 16 | 100.00 | ±.00 | 8.98 | ±.53 |
| L | 33 | 72.89 | ±.11 | 9.00 | ±.22 |
| M | 42 | 100.00 | ±.00 | 9.20 | ±.42 |
| N | 84 | 72.62 | ±.77 | 9.23 | ±.45 |
| O | 115 | 44.35 | ±.22 | 9.41 | ±.47 |
| P | 33 | 82.00 | ±.97 | 9.50 | ±.47 |
| Q | 18 | 100.00 | ±.00 | 9.70 | ±.62 |
| R | 42 | 76.19 | ±.09 | 10.13 | ±.59 |
| S | 116 | 43.10 | ±.33 | 10.18 | ±.46 |
| T | 10 | 100.00 | ±.00 | 10.25 | ±.74 |
| U | 121 | 100.00 | ±.00 | 10.88 | ±.25 |
| V | 55 | 100.00 | ±.00 | 10.98 | ±.37 |
| W | 44 | 100.00 | ±.00 | 11.00 | ±.47 |
| Mean | 15.62 ± 8.71 | 80.72 | ±24.58 | 8.93 | ±1.20 |

The data include a mean value and a standard error, based on each ejaculate.

To minimize the female variables, sows used in the present embodiment had 2 or more parities and inseminated twice per estrus by well-trained technicians. Although the number of inseminations in sows was small, the standard errors for farrowing rate and litter size were no higher in a group with few inseminations than in a group with many inseminations. Therefore, it was determined that all the possible variables had been minimized.

To calculate the accuracy of the optimized SPA and determine the normal range, semen samples from 24 Duroc boars were used. Whole semen samples from these boars were collected at weekly intervals by a gloved hand technique and filtered through cotton gauze into a pre-warmed flask at 25° C. All ejaculates showed motilities greater than 80%. Boar semen samples were diluted in BTS and kept at 17° C.

(3) Preparation and Treatment of Sperm Cells

The sperm samples were prepared by centrifugation at 500×g for 3 minutes and sperm pellets were diluted with PBS containing 0.4% bovine serum albumin (BSA). Subsequently, suspensions were centrifuged at 1200×g for 3 minutes and sperm pellets were treated with TYB and heparin. TYB was then added to the pretreated sperm pellets, and these pellets were incubated at 4° C. for 24 hours and then at 24° C. for 30 minutes. After incubation, sperm cells were centrifuged at 1200×g for 3 minutes, and sperm pellets were diluted to 2×10⁸ cells/ml in a TCM 199 medium. The sperm cells were then co-incubated with immature porcine oocytes and zona-free hamster oocytes.

The pretreated sperm pellets were mixed with a heparin medium and incubated at 39° C. for 30 minutes. After that, supernatants were centrifuged at 1200×g for 3 minutes, and sperm pellets were diluted to 2×10⁶ cells/ml in a heparin medium. The sperm cells were co-incubated with immature porcine oocytes and zona-free hamster oocytes.

(4) Preparation and Treatment of Sperm Cells for Quality Control

Two frozen bovine semen samples with medium fertility were used as internal controls. Individual straws, frozen at the same time, were assayed with the tested boar semen samples simultaneously over the experimental period. To evaluate the stability of the culture system, two frozen bovine semen samples were used. The sperm samples were pretreated by centrifugation at 1200×g for 3 minutes, and sperm pellets were diluted with PBS containing 4% BSA. Suspensions were centrifuged at 150×g for 3 minutes, and supernatants were then centrifuged at 1200×g for 3 minutes. Subsequently, sperm pellets were diluted to 2×10⁶ cells/ml in TCM 199 medium, and co-incubated with zona-free hamster oocytes at 39° C. for 3 hours.

### Exemplary Embodiment 2

### Sperm Penetration Assay Using Immature Porcine Oocytes

Porcine oocytes were collected from fresh ovaries of gilts (female pigs not experienced in farrowing) weighing approximately 95 kg right after slaughter at a local abattoir and transferred in about 30 minutes to a laboratory in 0.9% saline solution containing 100 ugl/ml streptomycin sulfate, from which oocytes were extracted.

Oocyte-cumulus complexes (COCs) were collected from 2 to 7 mm diameter follicles with an 18 gauge needle attached to a 10 ml syringe. The COCs were washed three times with modified DPBS before exposure to boar sperm cells. Each group of 15 immature oocytes was co-incubated with the sperm cells for 24 hours in a 4-well multidish containing 500 ul of each treatment medium at 39° C. in a 5% CO₂ atmosphere.

At the end of the co-incubation period, the cumulus cells and sperm cells were separated from the oocytes, which were mounted on slides and fixed for a minimum of 24 hours with a 3:1 mixture of ethanol and acetic acid. The oocytes were then stained with 1% lacmoid and examined for evidence of sperm penetration under a phase contrast microscope (×400).

### Exemplary Embodiment 3

### Sperm Penetration Assay Using Zona-Free Hamster Oocytes

Zona-free hamster oocytes were obtained from mature golden hamsters that had been administered on day 1 of their estrous cycle with PMSG and hCG (30 IU each) by intraperitoneal injection for 48 and 72 hours apart. The ovaries were excised, and the cumulus mass were removed from the excised oviducts and treated with 0.1% hyaluronidase and 0.1% trypsin to remove the cumulus cells and zona pellucida, respectively. The oocytes were washed three times with PBS after the enzyme treatment.

Each group of 10 hamster oocytes was co-incubated with sperm cells for 3 hours in a 4-well multidish containing 500 ul of each treatment medium at 39° C. in a 5% CO₂ atmosphere.

At the end of the co-incubation period, the oocytes were mounted on slides and fixed for a minimum of 24 hours with a 3:1 mixture of ethanol and acetic acid. The oocytes were then stained with 1% lacmoid and examined under a phase contrast microscope (×400) for evidence of sperm penetration.

### Exemplary Embodiment 4

### Calculation of Accuracy

The data obtained from the SPA were expressed as a sperm fertility index (SCl) (Johnson et al, 1995).

SCI={the mean number of penetrated sperm per ovum(the number of enlarged sperm heads+the number of decondensed sperm heads)+the number of pronuclei per ovum}/the number of ova

The present inventors also expressed the results as SFI as follows.

SFI(Sperm Fertility Index)={the mean number of penetrated sperm per ovum(the number of enlarged sperm heads+the number of decondensed sperm heads)+(the number of pronuclei per ovum×2)}/the number of ova

The SFI weighs the different scores according to their activation in the ovum. Therefore, the pronucleus was given with a two-fold higher score than the enlarged and decondensed sperm heads.

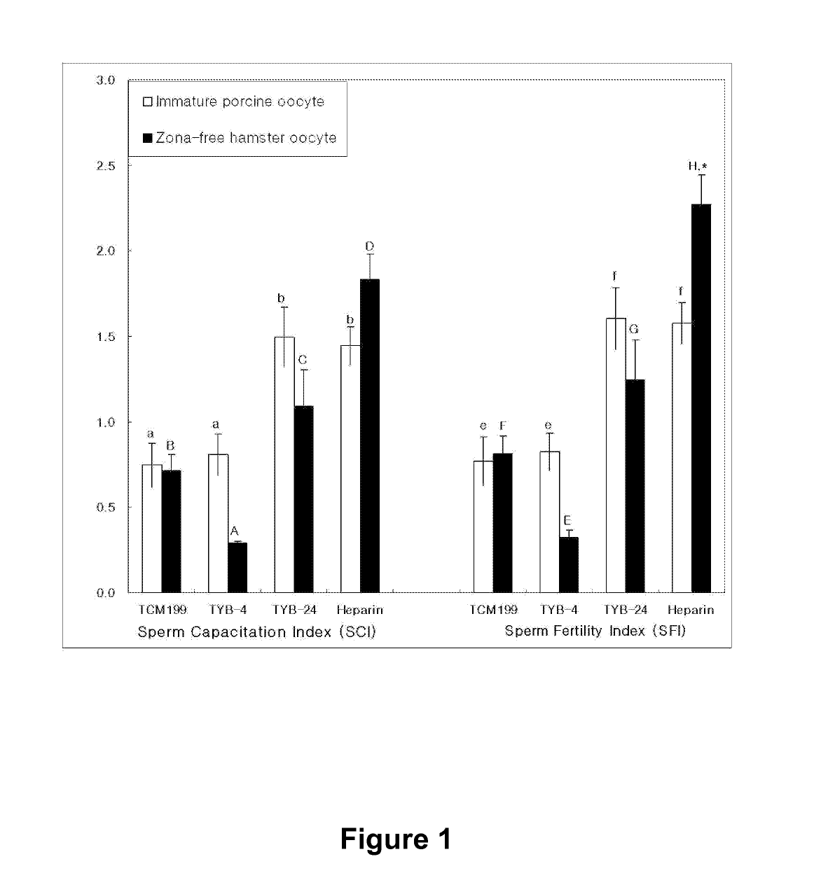

Four major parameters are involved in the evaluation test qualities: sensitivity, specificity, positive predictive value, and negative predictive value (Evans et al., 2002; FIG. 1).

Sensitivity determines percentage, which may be used to correctly identify all boars that can have either a small or large litter size. Specificity determines what percentage of the truly negative boars will test negative. It is important to know the percentage that actually have a small or large litter size of all boars having a positive test result, that is, the positive value. The negative predictive value is the percentage of the boars having a negative test result that actually have a small or large litter size.

Statistics

Statistical analyses were performed using a statistical software program (SPSS Version 12.0, USA). Comparison of the capacitiation methods with ovum sources was performed by one-way ANOVA. If a p value was <0.05 in the ANOVA, Turkey's HSD test was performed. All analyses of a quality control procedure were independently carried out using the chi-squared test. Pearson correlation coefficients were calculated to determine the association between litter size, farrowing rate and SFI.

Results

1. Optimization of Each Step of SPA

FIG. 1 shows effects of variables on the results of SPA. Data are expressed as mean SCI and SFI (±SE) by different capacitation methods and ovum sources (Eighty to ninety immature porcine oocytes and 30-33 zona-free hamster oocytes were used per treatment group in three individual experiments).

To increase the assay sensitivity, each step in the procedure was optimized. The goal of optimization was the establishment of conditions that led to maximum sperm penetration (wide penetration range).

The capacitation was induced by incubation in TCM 199 with or without heparin and low-temperature capacitation (4° C.) in TEST-yolk solution for 4 hours or 24 hours. The present inventors found that maximum penetration of zona-free hamster oocytes and immature porcine oocytes was obtained from heparin-treated sperm cells.

Zona-free hamster oocytes (2.267±0.176) showed a significantly higher penetration rate than immature porcine oocytes (1.576±0.122). To increase the discriminatory potential between good and poor fertility groups, the calculated using the SCI and the SFI were compared. The SFI showed a wider range of the spectrum, therefore, the present inventors expressed all data using the SFI from this point.

2. Quality Control

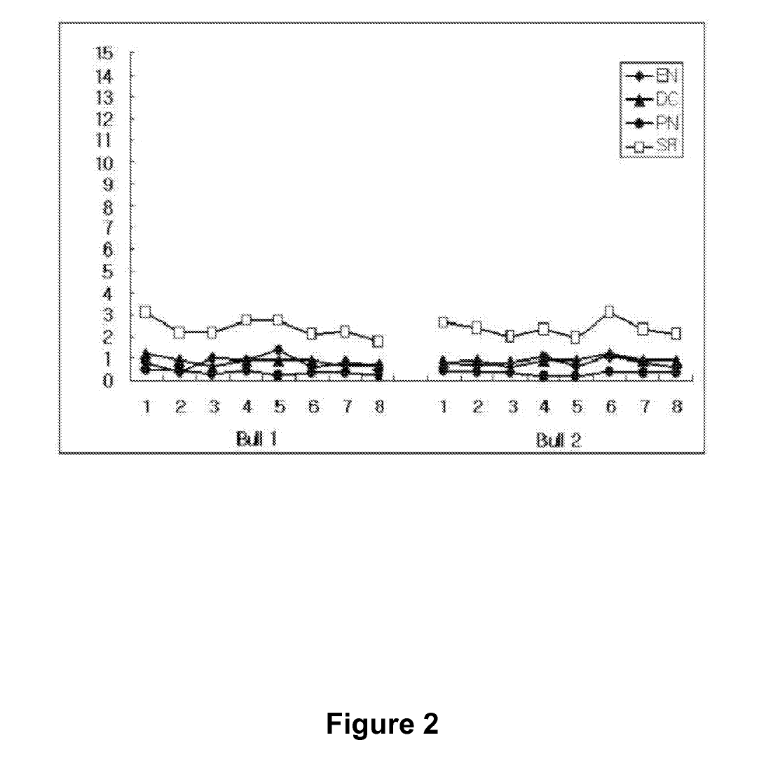

FIG. 2 shows a quality control plot for the penetration assay. Frozen bull sperm with a medium penetration rate was used to monitor interassay variation in each SPA assay throughout the entire experimental period (EN: Enlarged sperm, DC: Decondensed sperm, PN: Formed pronucleus and SFI: Sperm fertility index).

FIG. 2 shows that the quality control plot obtained from frozen bovine sperm with medium fertility that was used to develop a quality control system for the optimized SPA and to monitor interassay errors and stability. There were no differences in the numbers of enlarged sperm, decondensed sperm, formed pronuclei and SFI among all experimental trials for any bull. Therefore, the frozen bovine semen was proven to be a reliable standard control for the SPA. Only when all the control data deviated no more than slightly from the mean values did the present inventors consider the tested sperm data to be correct.

3. Relationship to Litter Size

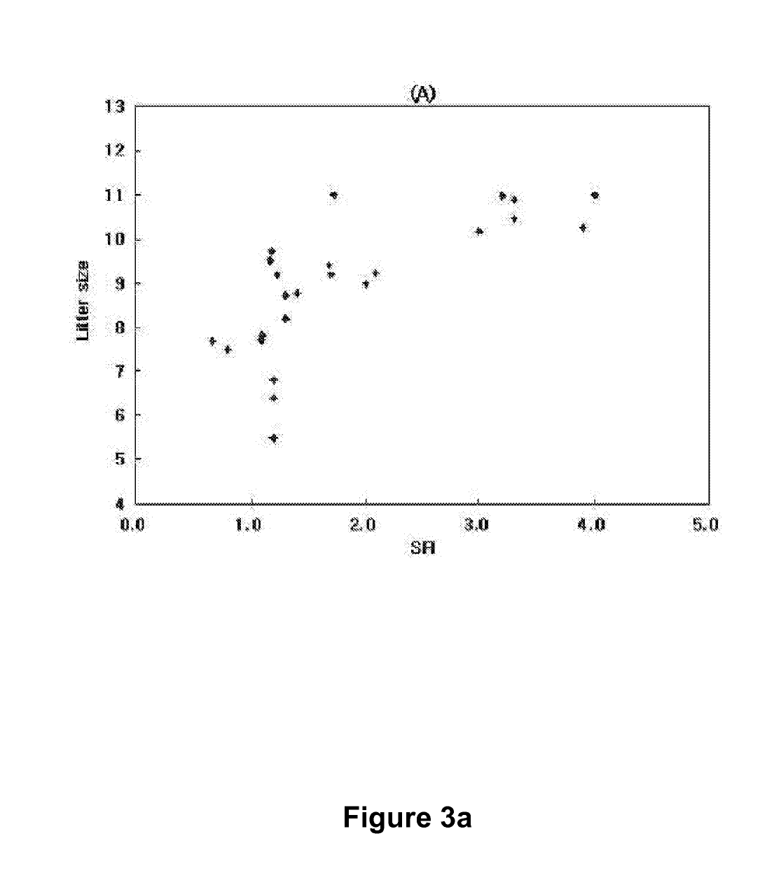

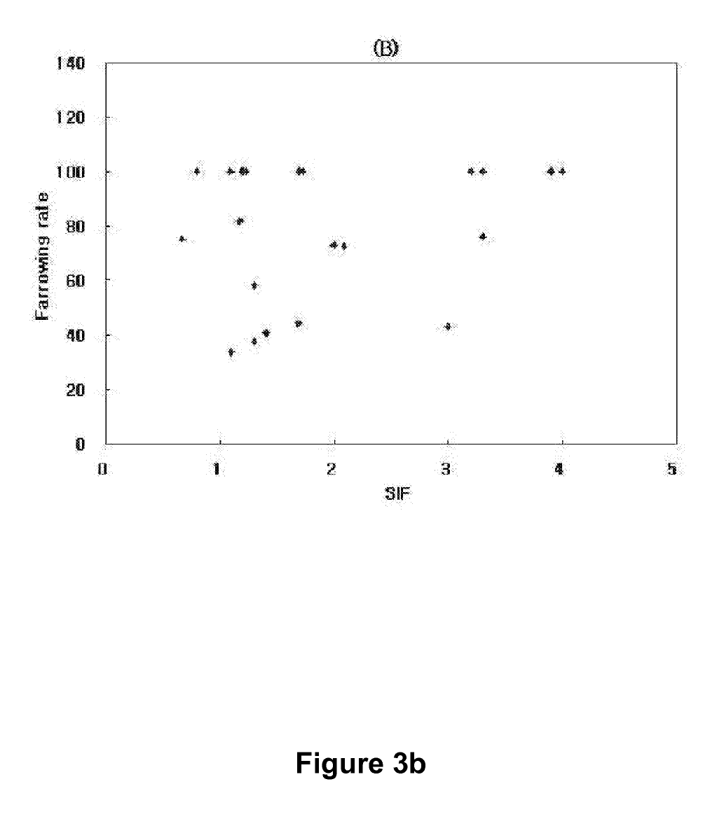

FIG. 3a shows a relationship of SPA to historic litter size. A significant correlation (r=0.726, p<0.05) was detected between SFI and litter size for the 24 boars.

FIG. 3b shows a relationship of SPA to historic farrowing rate. No significant correlation (r=0.140) was detected between SFI and farrowing rate.

That is, the SFI revealed a significant relationship to the historic average litter size (r=0.726, p<0.05), but not to the historic farrowing rates.

4. Determination of Normal Range with respect to Optimized SPA

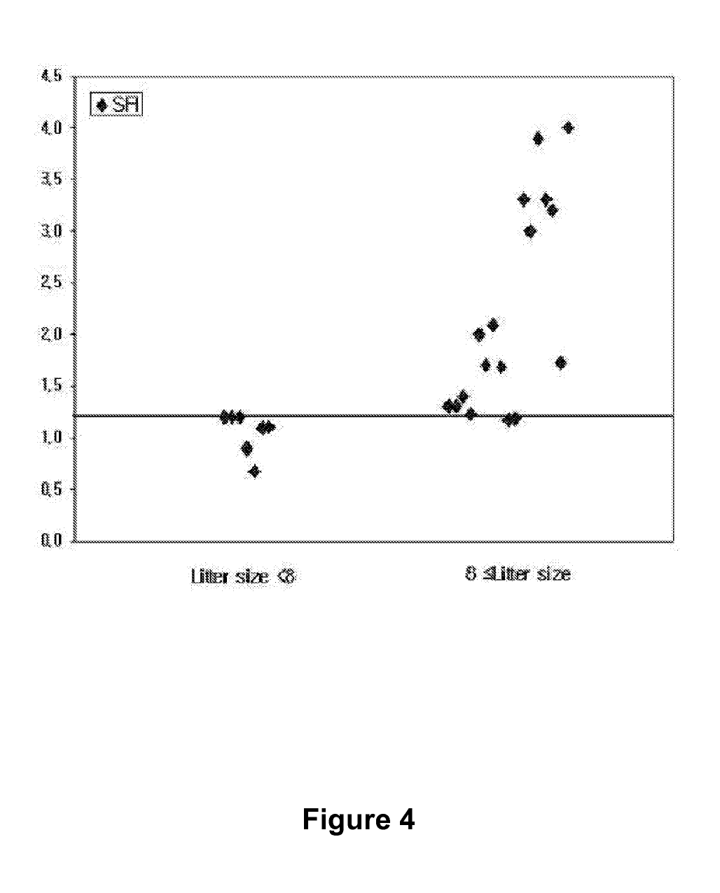

(1) FIG. 4 shows determination of the lower limit of the normal litter size for the optimized SPA. The lower limit of the normal litter size was defined as 8 or more.

Table 2 shows a correlation between SFI and litter size. Boars with an SFI of 1.2 or more have an increased probability of producing smaller litter size.

TABLE 2
Correlation between SFI and litter size

| | Litter size < 8 | Litter size = 8 |
|---|---|---|
| SFI = 1.2 (n = 9) | 7 | 2 |
| SFI > 1.2 (n = 15) | 0 | 15 |

| | |
|---|---|
| Sensitivity | 100% |
| Specificity | 88% |
| Positive predictive value | 78% |
| Negative predictive value | 100% |
| Accuracy | 92% |

To determine the normal range with respect to the optimized SPA, the lower limit of SFI was estimated as 1.2 with reference to FIG. 4.

Table 2 shows that a positive score (SFI≦1.2) in the SPA is highly predictive of the smaller litter size (<8). With a SFI score of 1.2 or more, 88% of the boars produced litters of fewer than eight piglets. In contrast, all the boars with a SFI score more than 1.2 had litters of eight or more piglets. Sensitivity indicated what percentage of the boars having smaller litter sizes would be identified by the test. Conversely, the specificity determined what percentage of the boars would test negative. Of all the boars that had a positive test result (SFI <1.2), it is important to know what percentage actually had the smaller litter size (<8), that is a positive predictive value. A negative predictive value is the opposite. Thus, it is the percentage of all the boars that had a negative test result (SFI>1.2) which actually had larger litter size (>8).

Accordingly, it is important that all of the boars with a litter of 8 or more have a SIF score of more than 1.2. It can be estimated that overall accuracy for prediction of the smaller litter size was 92%.

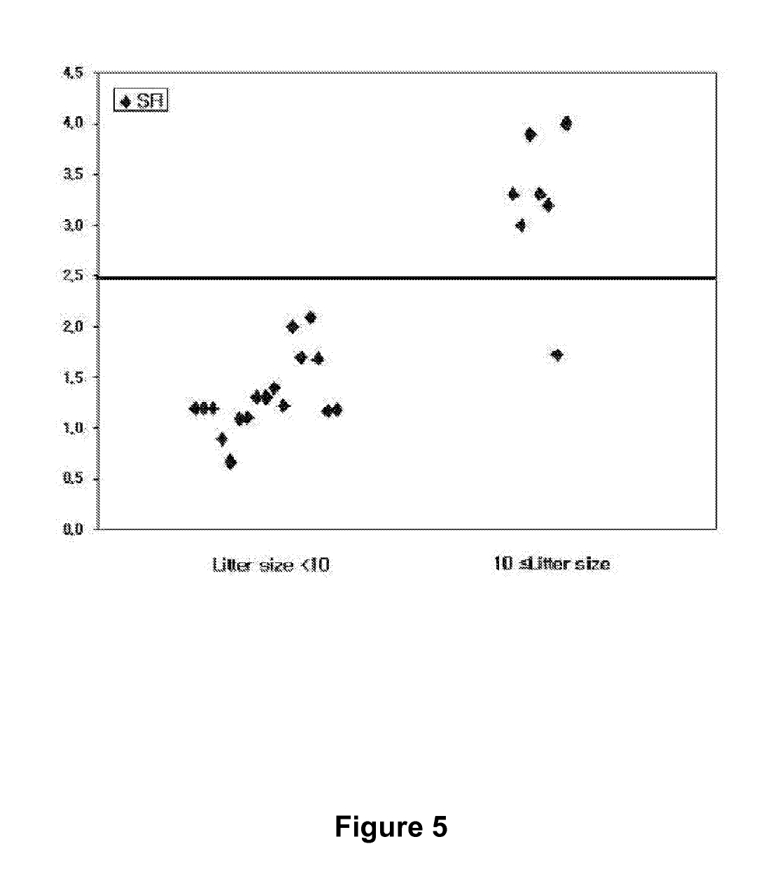

(2) FIG. 5 shows determination of the lower limit of the larger litter size for the optimized SPA. The lower limit of the larger litter size was defined as 10 or more in this case.

Table 3 shows a correlation between SFI and litter size. Boars with an SFI of more than 2.5 have an increased probability of larger litter size.

TABLE 3
Correlation Between SFI and Litter Size

| | Litter size = 10 | Litter size < 10 |
|---|---|---|
| SFI > 2.5 (n = 6) | 6 | 0 |
| SFI = 2.5 (n = 18) | 1 | 17 |

| | |
|---|---|
| Sensitivity | 86% |
| Specificity | 100% |
| Positive predictive value | 100% |
| Negative predictive value | 94% |
| Accuracy | 96% |

To determine the cut-off for larger litter size using the optimized SPA, SFI of 2.5 was established as the lower limit with reference to FIG. 5.

Table 3 shows that a positive score (SFI>2.5) in the SPA is highly predictive of the larger litter size (≧10). All the boars with a SFI score of more than 2.5 produced litters of ten or more piglets. In contrast, 94% of the boars with a SFI score of 2.5 or more had litters of less than ten piglets. It is important that 86% of the boars producing litters of ten or more piglets had a SFI score of more than 2.5. Overall accuracy for prediction of the larger litter size was 96%.

The present invention relates to a method of predicting pig litter size by optimizing a procedure for SPA, applying quality control and increasing sensitivity to discriminate between boars producing smaller and larger litters. Accordingly, boars can be discriminated based on their fertility and whether they produce larger or smaller litters. Thus, although the conditions for sperm capacitation and penetration are very different from those for an in vivo situation, meaningful information about sperm associated with fertility, acrosome reaction, fertilization and successful pregnancy can be obtained. Consequently, this method can improve breeding productivity and contribute to stable pig production.

Exemplary embodiments of the present invention have been disclosed herein and, although specific terms are employed, they are used and are to be interpreted in a generic and descriptive sense only and not for purpose of limitation. Accordingly, it will be understood by those of ordinary skill in the art that various changes in form and details may be made without departing from the spirit and scope of the present invention as set forth in the following claims.

What is claimed is:

1. A method of predicting boar sperm fertility, comprising:
incubating sperm cells from semen extracted from a boar in a heparin-treated medium;
co-incubating the incubated sperm cells with zona-free hamster oocytes in the heparin-treated medium; and
counting the numbers of penetrated sperm cells and pronuclei per ovum to analyze in-vitro sperm penetration.

2. The method according to claim 1, wherein the prediction of sperm fertility is performed by prediction of litter size.

3. The method according to claim 2, wherein the litter size is predicted as 8 or fewer piglets when SFI given by Formula I is equal to or smaller than 1.2
SFI={the mean number of penetrated sperm per ovum(the number of enlarged sperm heads+the number of decondensed sperm heads)+(the number of pronuclei per ovum×2)}/the number of ova. [Formula I]

4. The method according to claim 2, wherein the litter size is predicted as 10 or more piglets when SFI given by Formula I is more than 2.5
SFI={the mean number of penetrated sperm per ovum(the number of enlarged sperm heads+the number of decondensed sperm heads)+(the number of pronuclei per ovum×2)}/the number of ova. [Formula 1]

5. The method according to claim 1, further comprising:
simultaneously treating frozen bovine semen by the same procedure as described in claim 1 to be used as an internal control for the prediction of boar sperm fertility and to detect errors in the test.

6. The method according to claim 1, wherein the heparin-treated medium is formed by treating a medium supplemented with inactivated fetal calf serum, sodium pyruvate, D-glucose, calcium lactate, penicillin G and streptomycin sulfate with heparin.

7. The method according to claim 1, wherein the sperm cells are incubated at 39° C. for 30 minutes.

8. The method according to claim 1, wherein the sperm cells are diluted to a concentration of 2×10⁶ cells/ml in the heparin medium for co-incubation.

9. The method according to claim 1, wherein the sperm cells are co-incubated at 39° C. for 2 to 4 hours in a 5% CO₂ atmosphere.
